(19) United States
(12) Patent Application Publication
(10) Pub. No.: US 2011/0201860 A1
(43) Pub. Date: Aug. 18, 2011

(54) Process for conversion of alkanes to aromatics

(76) Inventors: Muhammad Naseem Akhtar, Dhahran (SA); Sachio Asaoka, Wakamatsu-ku (JP); Sulaiman S. Al-Khattaf, Dhahran (SA)

(21) Appl. No.: 12/656,900

(22) Filed: Feb. 18, 2010

(51) Int. Cl. C07C 2/00; B01J 29/068

(52) U.S. Cl. 585/419; 502/61

(57) ABSTRACT

The process for conversion of alkanes to aromatics includes the steps of contacting a feedstock containing alkanes having between two and six carbon atoms per molecule with a composite catalyst to produce an aromatization reaction, and collecting aromatics produced by the reaction. The composite catalyst is a zeolite having a matrix impregnated with a noble metal and an oxide of a transition metal. The noble metal may be Pt, Pd, Rh, Ru, or Ir. The transition metal may be Fe, Co, Ni, Cu, or Zn. The zeolite may be a medium or large pore zeolite, and may have an MFI, MEL, FAU, TON, VPI, MFL, AEI, AFI, MWW, BEA, MOR, LTL, or MTT structure, preferably MFI. The zeolite framework may include silicon, aluminum, and/or gallium. The matrix may be an oxide of magnesium, aluminum, titanium, zirconium, thorium, silicon or boron, and is preferably alumina.

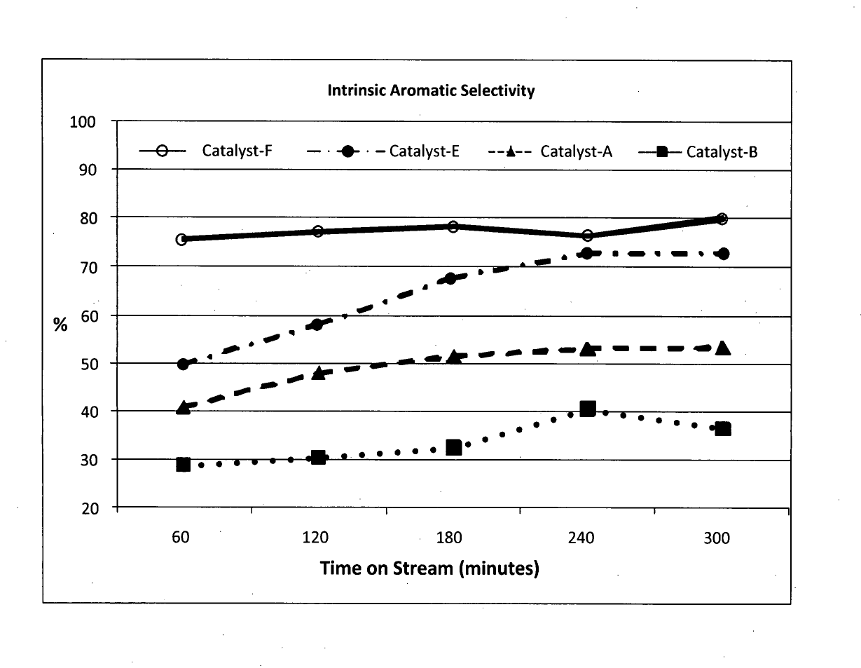

### BACKGROUND OF THE INVENTION

1. Field of the Invention

The present invention relates to catalytic processes for the conversion of alkanes having between two and six carbon atoms to aromatics, and particularly to a process for the conversion of alkanes to aromatics that uses a medium or large pore zeolite having a matrix containing a noble metal and an oxide of a transition metal.

2. Description of the Related Art

Aromatization is a well-known reaction in which alkanes are converted to aromatics. Aromatics, such as benzene, toluene, xylene (BTX) can be commercially produced by catalytic reforming of petroleum naphtha. However, naphtha is in great demand for other petrochemical products, such as gasoline.

One example of an aromatization process that does not use naphtha as feedstock is the Cyclar process, which converts liquefied petroleum gas (LPG) directly into aromatic products in a single operation. LPG mainly consists of propane and butane but can also contain C₂, Cₛ, and C₆ paraffins and C₂-C₆ olefins. LPG is primarily recovered from gas and oil fields and petroleum refining operations. LPG is relatively low in value and is available in abundance. These qualities make LPG a good feedstock for petrochemical applications, such as aromatization.

The Cyclar process is described as dehydro-cyclo-dimerization. This reaction is a sequential dehydrogenation of C₃ and/or C₄ alkanes to olefins, oligomerization of the olefins, cyclization of oligomeric products to naphthenes and dehydrogenation of naphthenes to corresponding aromatics. However, some side reactions, such as hydrocracking, isomerization, and dehydrogenation, also occur during aromatization. The typical catalyst used in the Cyclar process is a gallium-containing ZSM-5 zeolite.

A zeolite is a crystalline hydrated alumino silicate that may also contain other elements in the crystalline framework and/or deposited on its surface. The term “zeolite” includes not only aluminosilicates, but substances in which the aluminum is replaced by other trivalent elements and substances in which silicon is replaced by other tetravalent elements. A zeolite can be prepared by preparing an aqueous mixture of silicon oxide, aluminum oxide (and optionally, oxides of other trivalent or tetravalent elements), and then subjecting this mixture to a hydrothermal crystallization process to form zeolite crystals. The zeolite crystals are separated from the gel and are washed, dried and calcined.

It has been reported in the literature that whenever the ZSM-5 zeolite is used as a catalyst for the aromatization of propane, it produces large amount of C₁ (methane) with the aromatics. However, when a zeolite is impregnated with a noble metal, it results in the production of a large amount of C₂ (ethane or ethene) with the aromatics during the aromatization of propane, which may be recycled to the feedstock, if desired, for increased efficiency. Therefore, it is very important to measure the intrinsic selectivity for aromatics, rather than just the aromatic yield. The intrinsic selectivity for aromatics is calculated by dividing the sum of all aromatics produced by the process with the sum of all aromatics plus all cracking products.

Thus, a process for conversion of alkanes to aromatics solving the aforementioned problems is desired.

### SUMMARY OF THE INVENTION

The process for conversion of alkanes to aromatics includes the steps of contacting a feedstock containing alkanes having between two and six carbon atoms per molecule with a composite catalyst to produce an aromatization reaction, and collecting aromatics produced by the reaction. The composite catalyst is a zeolite having a matrix impregnated with a noble metal and an oxide of a transition metal. The noble metal may be Pt, Pd, Rh, Ru, or Ir. The transition metal may be Fe, Co, Ni, Cu, or Zn. The zeolite may be a medium or large pore zeolite, and may have an MFI, MEL, FAU, TON, VPI, MFL, AEI, AFI, MWW, BEA, MOR, LTL, or MTT structure, preferably MFI. The zeolite framework may include silicon, aluminum, and/or gallium. The matrix may be an oxide of magnesium, aluminum, titanium, zirconium, thorium, silicon or boron, and is preferably alumina.

The process may include the step of contacting the feedstock with the catalyst at a space hour velocity between 0.1 and 10,000 (hr⁻¹), preferably between 1.0 and 5,000 (hr⁻¹). The process may further include the step of contacting the feedstock with the catalyst at a temperature in the range of 200 to 600° C., preferably 300 to 600° C. The process may further include the step of contacting the feedstock with the catalyst at a pressure in the range of 1.0 to 10.0 bars.

These and other features of the present invention will become readily apparent upon further review of the following specification and drawing.

### BRIEF DESCRIPTION OF THE DRAWINGS

The sole drawing FIGURE is a plot of intrinsic aromatic selectivity against time on stream in for four exemplary catalysts.

### DETAILED DESCRIPTION OF THE PREFERRED EMBODIMENTS

The process for conversion of alkanes to aromatics includes the steps of contacting a feedstock containing alkanes having between two and six carbon atoms per molecule with a composite catalyst to produce an aromatization reaction, and collecting aromatics produced by the reaction. The composite catalyst includes a zeolite and a matrix impregnated with at least one oxide of a transition metal from the group made up of Fe, Co, Ni, Cu, and Zn, and a metal from the noble metal group, such as Pt, Pd, Rh, Ru, and Ir. The catalyst is used to convert C₂-C₆ alkanes to aromatics, such as benzene, toluene and xylenes.

The structure of the zeolite may be MFI, MEL, FAU, TON, VPI, MFL, AEI, AFI, MWW, BEA, MOR, LTL or MTT, but is preferably MFI, having gallium and/or aluminum and silicon in the frame work. The Ga—Al—Si zeolite is synthesized from an aqueous gel containing a silica source, a gallium source, an aluminum source and a structure-directing agent. The typical technique for synthesizing the Ga—Al—Si zeolite comprises converting an aqueous gel of a silica source, a gallium source and an aluminum source to zeolite crystals by a hydrothermal process employing a dissolution/recrystallization mechanism. The reaction medium may also contain structuring agents, which are incorporated into the microporous space of the zeolite network during crystallization, thus controlling the construction of the network and assisting to stabilize the structure through the interactions with the zeolite components. The reaction mixture gel is heated and stirred to form zeolite crystals and then cooled. The zeolite crystals are separated from the gel and are washed, dried and calcined.

The silicon-to-aluminum and/or gallium atomic ratio [Si/(Ga+Al)] of the zeolite is preferably greater than 2. One example of an acceptable ratio for the zeolite framework is a [Si/(Ga+Al)] atomic ratio in the range from 10 to 200. Also acceptable is a [Si/(Ga+Al)] atomic ratio in the range from 20 to 150. It will be understood that the foregoing ranges are exemplary, and not intended to be limiting.

The zeolite is a medium pore zeolite or large pore zeolite. The term “medium pore” refers to an average pore size of five to about seven angstroms. The term “large pore” refers to an average pore size of seven to about ten angstroms. It is possible that these ranges could overlap, and a particular zeolite might be considered either a medium pore zeolite or a large pore zeolite. Zeolites having an average pore size of less than about five angstroms, i.e., a “small pore” zeolite, would not be considered either a medium pore zeolite or a large pore zeolite. A small pore zeolite would not allow molecular diffusion of the molecules of the desired aromatic products, e.g., benzene, ethylbenzene, toluene and xylenes, in its pores and channels. Examples of medium pore zeolites and large pore zeolites suitable for use in the composite catalyst are MFI, MEL, FAU, TON, VPI, MFL, AEI, AFI, MWW, BEA, MOR, LTL or MTT.

The matrix in this composite catalyst system includes at least one oxide of a metal from the group made up of magnesium, aluminum, titanium, zirconium, thorium, silicon and boron. The preferred matrix is alumina with a surface area from 10-600 m²/g, and preferably from 150-400 m²/g. The matrix is impregnated with at least one oxide of a transition metal from the group made up of Fe, Co, Ni, Cu, and Zn, and a metal from the noble metal group, such as Pt, Pd, Rh, Ru, and Ir.

The composite catalyst of the invention may be prepared by two methods, which are described theoretically below.

In a first method, the zeolite is mixed with the matrix. The mixture can be structured by any of the processes described in the prior art, such as: pelleting, extrusion, tableting, and coagulation in drops or spray drying. After structuring, at least one oxide of a transition metal from the group made up of Fe, Co, Ni, Cu, and Zn is deposited first, then a metal from the noble metal group, such as Pt, Pd, Rh, Ru, or Ir, is deposited. Typical methods of depositing a metal or metal oxide are ion exchange and impregnation. The at least one oxide of a transition metal from the group made up of Fe, Co, Ni, Cu, and Zn and a metal from the noble metal group, such as Pt, Pd, Rh, Ru, or Ir, is then deposited on the matrix. The oxide of a transition metal from the group made up of Fe, Co, Ni, Cu, and Zn can be 0.1-50% by weight, preferably from 1-30%. The metal from the noble metal group of Pt, Pd, Rh, Ru, or Ir may be 0.01-20% by weight, and is preferably from 0.01-10%.

In a second method, the oxide of a transition metal from the group made up of Fe, Co, Ni, Cu, and Zn and a metal from the noble metal group consisting of Pt, Pd, Rh, Ru, and Ir are impregnated on the matrix. Typical methods for impregnation of a metal or metal oxide are ion exchange and impregnation. The zeolite is then mixed with the matrix already impregnated with the at least one oxide of a transition metal from the group made up of Fe, Co, Ni, Cu, and Zn and a metal from the noble metal group consisting of Pt, Pd, Rh, Ru, and Ir, and is then structured.

The preferred method for preparation of the composite catalyst is based on the second method, in which the matrix is first impregnated with the metal oxide and the noble metal, and is then mixed with the zeolite and structured.

The catalyst may be used in a process of aromatization of alkanes, such as alkanes having two to six carbon atoms per molecule, to produce aromatics, such as benzene, toluene and xylene (BTX). The catalyst is pre-activated by reduction with hydrogen before aromatization. The catalyst is reduced with hydrogen at a temperature range of 350 to 600° C. from 1 to 24 hours. The aromatization reaction of the alkane may be carried out at a temperature in the range between 200 to 600° C., preferably between 300 to 600° C., and at a pressure in the range between 1 and 10 bars. The contact between the alkane and the catalyst is at a space hour velocity in the range of 0.1 to 10,000 (hr⁻¹), preferably in the range of 1.0 to 5,000 (hr⁻¹).

The following examples are provided to show the process of preparing and using the composite catalyst generally by way of illustration, and not for purposes of limitation, as well as catalysts not of this invention, but used as comparative examples.

### EXAMPLE 1

### Synthesis of Ga—Al-Silicate (MFI) Zeolite

Two separate solutions named as solution-A and solution-B were prepared for synthesis of the Ga—Al-silicate (MFI) zeolite. Solution-A was prepared by adding 33.41 g of de-ionized water to 25.59 g of sodium silicate solution (SiO₂, 29% by weight) in a beaker. Solution-B was prepared by taking 44.64 g of water in a beaker. Then, 1.77 g of Al₂(SO₄)₃. 14˜18H₂O, 0.65 g of Ga(NO₃)₃.nH₂O, 1.82 g of concentrated H₂SO₄ (98%), 4.90 g of NaCl and 9.41 g of tetrapropyl ammonium bromide (TPABr) were added one-by-one to the water with continuous stirring to obtain a clear solution.

Solution-B was then added to solution-A drop-by-drop while stirring continuously with a gel mixer at high speed. A thick viscous gel was formed, which was added to the Teflon liner beaker of a metallic cylinder. The metallic cylinder was sealed tightly and was connected to a metallic shaft inside an oven. The oven was heated to 150° C., and the shaft was rotated at 14 revolutions-per-minute. The mixture was treated under these hydrothermal conditions for 24 hours. The mixture was cooled down using water at room temperature to quench the crystallization process. The resulting zeolite powder was filtered and washed with plenty of distilled water 10-12 times until the pH of the filtrate came down from 12 to 7. The zeolite powder was dried in an oven at 120° C. for 3 hours and calcined at 550° C. for 3 hours.

The MFI structure of the zeolite was confirmed by measuring the powder X-Ray diffraction pattern. Elemental analysis was performed using XRF. The Al—Ga-Silicate (MFI) zeolite prepared in this way had a ratio of [Ga/(Al+Ga)]=0.2 and [Si/(Al+Ga)]=14.0.

### EXAMPLE 2

### Preparation of Al—Ga-Silicate-Al₂O₃ (Catalyst-A)

Na—Al—Ga-Silicate was structured into extrudates by mixing 6.0 g of Al—Ga-Silicate with 4.0 g of very fine alumina having a pore size distribution of 4 nm. The extrudates were dried at 120° C. for three hours and calcined at 550° C. for three hours. The extrudates of Na—Al—Ga-Silicate-Al₂O₃ were then ion exchanged with aqueous solution of ammonium nitrate. The 5.0 g of Na—Al—Ga-Silicate-Al₂O₃ was refluxed with 25.0 ml of 2.2M ammonium nitrate aqueous solution for 2 hours. Then the extrudates were filtered and washed with plenty of deionized water. The extrudates were refluxed four times with ammonium nitrate solution using this procedure. The extrudates were then dried at 120° C. for three hours and calcined at 550° C. for three hours to yield a comparative example designated as catalyst-A.

### EXAMPLE 3

### Preparation of Pt/Al—Ga-Silicate-Al₂O₃ (Catalyst-B)

The ion exchange impregnation method was used to load platinum on catalyst-A (Al—Ga-Silicate-Al₂O₃).

A 0.053 g sample of H₂PtCl₆ was dissolved in 20 ml of water and added to 4.0 g of catalyst-A (Al—Ga-Silicate-Al₂O₃). The mixture was left overnight at room temperature to equilibrate. Then water was removed from the mixture using a rotary evaporator. The catalyst was then dried at 120° C. for three hours and calcined at 550° C. for three hours.

The catalyst prepared in this way, designated catalyst-B [Pt/Al—Ga-Silicate-Al₂O₃], contained 0.5% platinum by weight.

### EXAMPLE 4

### Preparation of Alumina Containing Zinc Oxide and Platinum (Catalyst-C)

The alumina used in this catalyst was very fine, having a pore size distribution of 4 nm, a surface area of 310 m²/g, and a pore volume of 0.46 cm³/g.

The alumina was first structured into extrudates. The ion exchange impregnation method was used to load Zinc oxide and platinum on the alumina. A 0.920 g sample of [Zn(NO₃)₂.6H₂O] was dissolved in 20 ml of water and added to the 5.0 g of alumina. The mixture was left overnight at room temperature to equilibrate. Then water was removed from the mixture using a rotary evaporator. The catalyst was then dried at 120° C. for three hours and calcined at 550° C. for three hours.

The calcined product containing Zinc oxide [ZnO/Al₂O₃ (4 nm)] was then impregnated with platinum. A 0.053 g sample of H₂PtCl₆ was dissolved in 20 ml of water and was added to the 4.0 g of [ZnO/Al₂O₃ (4 nm)]. The mixture was left overnight at room temperature to equilibrate. Then water was removed from the mixture using a rotary evaporator. The catalyst was then dried at 120° C. for three hours and calcined at 550° C. for three hours.

The catalyst prepared in this way, designated as catalyst-C [Pt/ZnO/Al₂O₃ (4 nm)], had 5.0% zinc oxide and 0.5% platinum by weight.

### EXAMPLE 5

### Preparation of Alumina Containing Zinc Oxide and Platinum (Catalyst-D)

The alumina used in this catalyst had a pore size distribution of 11 nm, a surface area of 305 m²/g, and a pore volume of 0.73 cm³/g.

The ion exchange impregnation method was used to load zinc oxide and platinum on the alumina. A 7.50 g sample of [Zn(NO₃)₂.6H₂O] was dissolved in 40 ml of water and was added to 10.0 g of alumina. The mixture was left overnight at room temperature to equilibrate. Then water was removed from the mixture using a rotary evaporator. The catalyst was then dried at 120° C. for three hours and calcined at 550° C. for three hours.

The calcined product containing zinc oxide [ZnO/Al₂O₃ (11 nm)] was then impregnated with platinum. A 0.106 g sample of H₂PtCl₆ was dissolved in 40 ml of water and was added to the 8.0 g of [ZnO/Al₂O₃ (11 nm)]. The mixture was left overnight at room temperature to equilibrate. The water was removed from the mixture using a rotary evaporator. The catalyst was then dried at 120° C. for three hours and calcined at 550° C. for three hours.

The catalyst prepared in this way, designated catalyst-D, had 20.0% zinc oxide and 0.5% platinum by weight.

### EXAMPLE 6

### Reduction of Catalyst-C

Catalyst-C [Pt/ZnO/Al₂O₃ (4 nm)] was reduced using hydrogen at a temperature of 450° C. for three hours. The gas hour space velocity (GHSV) of hydrogen was adjusted to 5000 (hr⁻¹).

### EXAMPLE 7

### Reduction of Catalyst-D

Catalyst-D [Pt/ZnO/Al₂O₃ (11 nm)] was reduced using hydrogen at a temperature of 450° C. for three hours. The gas hour space velocity (GHSV) of hydrogen was adjusted to 5000 (hr⁻¹).

### EXAMPLE 8

### Preparation of Pt/ZnO/Al₂O₃ (4 nm)+Al—Ga-Silicate-Al₂O₃ (4 nm)+SiO₂ (Catalyst-E)

The reduced catalyst-C of Example 6 was crushed into powder form and mixed with reference catalyst-A.

A 2.0 gram sample of reduced Catalyst-C [Pt/ZnO/Al₂O₃ (4 nm)] was mixed with 2.0 g of reference catalyst-A [Al—Ga-Silicate-Al₂O₃ (4 nm)] and 1.0 g of silica binder. The whole mixture was then structured into extrudates. The extrudates were dried at 120° C. for three hours and calcined at 550° C. for three hours.

### EXAMPLE 9

### Preparation of Pt/ZnO/Al₂O₃ (11 nm)+Al—Ga-Silicate-Al₂O₃ (4 nm)+SiO₂ (Catalyst-F)

The reduced catalyst-D of Example 7 was crushed into powder form and mixed with reference catalyst-A.

A 2.0 gram sample of reduced Catalyst-D [Pt/ZnO/Al₂O₃ (11 nm)] was mixed with 2.0 g of reference catalyst-A [Al—Ga-Silicate-Al₂O₃ (4 nm)] and 1.0 g of silica binder. The whole mixture was then structured into extrudates. The extrudates were dried at 120° C. for three hours and calcined at 550° C. for three hours.

### EXAMPLE 10

### Catalyst Evaluation

The catalysts prepared by the above procedures were tested for aromatization of propane. The catalysts were reduced in-situ before the aromatization reaction.

The catalyst extrudates were crushed and sieved to get the particle size in the range of 355-600 μm. A 2.0 ml sample of catalyst particles was loaded in the centre of a tubular reactor by placing neutral glass beads above and below the catalyst bed. The catalyst was first reduced by hydrogen gas at a temperature of 450° C. for three hours with a gas hour space velocity (GHSV) of 5000 (hr⁻¹).

The aromatization of propane was carried out at 538° C. with a gas hour space velocity (GHSV) of 1500 (hr⁻¹) under atmospheric pressure. The reactor was fed with a mixture of propane and nitrogen in a ratio of 1:2. The reaction products were analyzed by gas chromatography.

The intrinsic selectivity for aromatics reported was calculated as the sum of all aromatics produced divided by the sum of all aromatics plus the sum of C₁, C₂, and C₂ olefin materials recovered.

The data in Table 1 shows a significant increase in intrinsic aromatic selectivity for composite catalyst-E and composite catalyst-F of the present invention as compared to reference catalyst-A and reference catalyst-B. It also shows a significant drop in C₁ and C₂ products for composite catalyst-E and composite catalyst-F of the present invention as compared to reference catalyst-A and reference catalyst-B

TABLE 1

| | | Intrinsic Aromatic |
|---|---|---|
| Catalyst | C₁, C₂, & C₂″ (%) | Selectivity (%) |
| Catalyst-A | 25 | 49 |
| Catalyst-B | 43 | 34 |
| Catalyst-E | 16 | 64 |
| Catalyst-F | 8 | 78 |

The intrinsic aromatic selectivity for all four catalysts has been plotted against time on stream in the sole drawing FIGURE. The FIGURE clearly shows a significant increase for both catalyst-E and catalyst-F as compared to reference catalyst-A and reference catalyst-B. Catalyst-F exhibited very high and stable intrinsic aromatic selectivity as compared to all other catalysts.

It is to be understood that the present invention is not limited to the embodiments described above, but encompasses any and all embodiments within the scope of the following claims.

We claim:

1. A process for conversion of alkanes to aromatics, comprising the steps of:
contacting a feedstock containing alkanes having between two and six carbon atoms per molecule with a composite catalyst to produce an aromatization reaction; and
collecting aromatics produced by the reaction;
wherein the composite catalyst is a zeolite having a matrix impregnated with a noble metal selected from the group consisting of platinum, palladium, rhodium, ruthenium, and iridium, and an oxide of a transition metal selected from the group consisting of iron, cobalt, nickel, copper, and zinc.

2. The process for conversion of alkanes to aromatics according to claim 1, wherein said zeolite has at least a medium pore size.

3. The process for conversion of alkanes to aromatics according to claim 1, wherein said zeolite has a structure selected from the group consisting of MFI, MEL, FAU, TON, VPI, MFL, AEI, AFI, MWW, BEA, MOR, LTL, and MTT.

4. The process for conversion of alkanes to aromatics according to claim 1, wherein said zeolite has a framework containing silicon and aluminum.

5. The process for conversion of alkanes to aromatics according to claim 1, wherein said zeolite has a framework containing silicon and gallium.

6. The process for conversion of alkanes to aromatics according to claim 1, wherein said zeolite has a framework has an atomic ratio of silicon to aluminum plus gallium greater than two.

7. The process for conversion of alkanes to aromatics according to claim 1, wherein said zeolite is an MFI zeolite having a framework containing silicon, aluminum, and gallium.

8. The process for conversion of alkanes to aromatics according to claim 1, wherein said matrix comprises alumina.

9. The process for conversion of alkanes to aromatics according to claim 8, wherein said matrix is impregnated with platinum and zinc oxide.

10. The process for conversion of alkanes to aromatics according to claim 9, wherein said zeolite has a framework containing silicon, aluminum, and gallium.

11. The process for conversion of alkanes to aromatics according to claim 10, wherein said alumina matrix has an average pore diameter between about 3 nm and about 6 nm.

12. The process for conversion of alkanes to aromatics according to claim 10, wherein said alumina matrix has an average pore diameter of about 8 nm and about 14 nm.

13. The process for conversion of alkanes to aromatics according to claim 1, wherein said contacting step further comprises the step of contacting the feedstock with the catalyst at a space hour velocity between 1.0 and 5,000 (hr⁻¹).

14. The process for conversion of alkanes to aromatics according to claim 1, wherein said contacting step further comprises the step of contacting the feedstock with the catalyst at a temperature between 300 and 600° C.

15. The process for conversion of alkanes to aromatics according to claim 1, wherein said contacting step further comprises the step of contacting the feedstock with the catalyst at a pressure between 1.0 and 10.0 bars.

16. A process for conversion of hydrocarbons to aromatics, comprising the steps of:
contacting a feedstock containing hydrocarbons having between two and six carbon atoms per molecule with a composite catalyst to produce an aromatization reaction; and
collecting aromatics produced by the reaction;
wherein the composite catalyst is a zeolite having:
a framework containing silicon, gallium, and aluminum in an atomic range of silicon to aluminum and gallium; and
an alumina matrix having an average pore diameter between about 3 nm and about 14 nm, the matrix being impregnated with platinum and zinc oxide.

17. The process for conversion of hydrocarbons to aromatics according to claim 16, further comprising the step of reducing said composite catalyst by hydrogen gas before said contacting step.

18. The process for conversion of hydrocarbons to aromatics according to claim 16, wherein said contacting step further comprises the step of contacting the feedstock containing the hydrocarbons with said composite catalyst at a temperature between about 490° C. and about 590° C. with a gas hour space velocity (GHSV) between about 1000 (hr⁻¹) and about 2000 (hr⁻¹) at a pressure of less than 2 bars.

19. A composite catalyst for conversion of hydrocarbons to aromatics, comprising a zeolite having:
a medium or large pore size framework containing silicon, aluminum, and gallium with the silicon being present in a ratio between 20 and 150 to the gallium and aluminum; and
an alumina matrix impregnated with a noble metal selected from the group consisting of platinum, palladium, rhodium, ruthenium, and iridium, and an oxide of a transition metal selected from the group consisting of iron, cobalt, nickel, copper, and zinc.

20. The composite catalyst according to claim 19, wherein said noble metal comprises platinum and said oxide of a transition metal comprises zinc oxide.
